(19) United States
(12) Patent Application Publication
(10) Pub. No.: US 2007/0083118 A1
(43) Pub. Date: Apr. 12, 2007

(54) Method and System For Estimating Cardiac Ejection Volume Using Ultrasound Spectral Doppler Image Data

(75) Inventor: Praveen Dala-Krishna, Sicklerville, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MEDSYSTEMS, INC., West Berlin, NJ (US)

(21) Appl. No.: 11/610,902

(22) Filed: Dec. 14, 2006

Related U.S. Application Data
(63) Continuation-in-part of application No. 11/428,517, filed on Jul. 3, 2006.
Application No. 11/428,517 is a continuation of application No. 10/620,517, filed on Jul. 16, 2003.
(60) Provisional application No. 60/397,653, filed on Jul. 22, 2002.

(51) Int. Cl. A61B 8/00

(52) U.S. Cl. 600/437; 600/443

(57) ABSTRACT

A method and system for estimating the volume of blood ejected from a cardiac ventricle or atrium uses spectral Doppler ultrasound while imaging a portion of the heart. The method computes the mean ejection velocity V_{avg}(t) at time t from the spectral Doppler data. The process may further utilize a discrete graphical technique to compute a measure of cardiac output. Further, the measure of cardiac output can yield an approximation to the mean-ejection-velocity integral, which is the area under the curve V_{avg}(t) for all moments t within a cardiac cycle.

### RELATED APPLICATIONS

This application is a continuation-in-part of U.S. patent application Ser. No. 11/428,517 filed Jul. 3, 2006, which is a continuation of U.S. patent application Ser. No. 10/620,517 filed Jul. 16, 2003, now U.S. Pat. No. ______, which claims the benefit of priority to U.S. Provisional Application Ser. No. 60/397,653, filed on Jul. 22, 2002, the entire contents of all of which previous applications are hereby incorporated by reference.

### FIELD OF THE INVENTION

The specification herein relates to a technique for measuring cardiac output (i.e., total volume of blood ejected by the left ventricle in one cardiac cycle) using Doppler ultrasonic imaging, particularly for use in evaluating placement of cardiac pacing electrodes.

### BACKGROUND OF THE INVENTION

Volumetric output of blood from the heart and/or circulatory system is of interest in various diagnostic and therapeutic procedures. Such measurements are of significant interest during electrophysiological evaluation/therapy to first evaluate the extent of dysfunction due to arrhythmia and subsequently to judge the effect/effectiveness of any ablations/therapeutic procedures that are carried out on the cardiac muscle/conduction system. Iwa et al., Eur. J. Cardithorac. Surg., 5, 191-197 (1991).

Ultrasound is the imaging modality of choice, especially in cardiology, since this modality offers real-time imaging capabilities of the moving heart. Further, advances through Doppler techniques allow the physician to visualize as well as measure blood flow. Pulse wave and continuous wave Doppler have proven to be quite accurate, and an effective way of evaluating flow through various parts of the circulatory system, especially the heart. Tortoli et al., Ultrasound Med. Bio., 28, 249-257 (2002); Mohan et al., Pediatr. Cardiol. 23, 58-61 (2002); Ogawa et al., J. Vasc. Surg., 35, 527-531 (2002); Pislaru et al., J. Am. Coll. Cardiol., 38, 1748-1756 (2001).

Other technologies, including washout curves of contrast agents have been proposed to measure flow volume, especially to compensate for loss of signal quality due to imaging depth. Krishna et al., Ultrasound Med. Bio., 23, 453-459 (1997); Schrope et al., Ultrasound Med. Bio., 19, 567-579 (1993).

However, until recent advances in miniaturized ultrasonic transducers, physicians were limited to only certain angles of view, thus limiting the range and effectiveness of possible measurements. Further, given the depth of imaging required by such classical approaches, associated interrogation frequency limitations due to attenuation restricted the accuracy of measurement. Krishna et al., Phys. Med. Biol., 44, 681-694 (1999). With the recent introduction of catheter based transducers for imaging the heart from either the vena-cava or even from within the heart, such limitations on frequency of interrogation and angle of view are not applicable.

One specific need for this invention is for the permanent placement of cardiac pacing electrodes. Cardiac pacing has been around for many years, and essentially involves the placement of a permanent electrode in the right ventricle to quicken the pace of an otherwise slow heart. A new therapy has recently been introduced to the market, which involves pacing of the left ventricle in conjunction with the right ventricle in an effort to “resynchronize” the heart, that is, to coordinate the left ventricle's contraction in time with the contraction of the right ventricle. One problem in the current therapy is the optimization of the placement of the left ventricular electrode so as to provide maximum therapy. This invention addresses this problem by providing intracardiac ultrasound imaging and ultrasound Doppler as a new tool in the placement of the electrode.

### SUMMARY OF THE INVENTION

An embodiment utilizes spectral Doppler ultrasound images to measure cardiac output and changes in volume from one cardiac cycle to the next. Such Doppler based embodiment can include hardware and/or software, either on the ultrasound system, or on a separate system that directly or indirectly communicates with or receives data from the ultrasound system and a device that can digitize and/or transmit ECG data, if separate from the ultrasound unit. Embodiment methods compute the mean ejection velocity V_{avg}(t) at time t from the spectral Doppler data, and may use a discrete graphical technique to compute a measure of cardiac output. Further, the measure of cardiac output can yield an approximation to the mean-ejection-velocity integral as the area under the curve V_{avg}(t) for all moments t within a cardiac cycle. Embodiment methods compute the mean ejection velocity V_{avg}(t) at time t from the spectral Doppler frequency shift data, and may use a discrete graphical technique to compute a measure of cardiac output. Further, the measure of cardiac output can yield an approximation to the mean-ejection-velocity integral as the area under the curve V_{avg}(t) for all moments t within a cardiac cycle. The various embodiments can utilize ultrasound data, in coordination with the ECG signals, to calculate the ejection volumes of the ventricle in the course of multiple cardiac cycles.

With the ability to measure cardiac ejection volumes and compare cycle-to-cycle variation, the effectiveness of a given therapy may be evaluated quantitatively. In particular, a clinician can evaluate the effectiveness of implanting a pacemaker electrode at a given cardiac location with the pacemaker programmed according to specific configuration parameters. By repeating the evaluation for more electrode locations and/or other specific pacemaker configuration parameters, the clinician can find an optimal combination of electrode location and pacemaker configuration. For the situation of comparing the relative effectiveness of two pacemaker configurations or electrode locations, the ejection volume area may be ignored when it does not change. Then the values of velocity-time integral may suffice when comparing the relative ejection effectiveness. The velocity-time integral may similarly suffice when comparing an image of the heart without a pacemaker with an image with one. The same technique may be used if the area does differ between two spectral images.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain features of the invention.

FIG. 1 provides a general system diagram showing an ultrasound system.

FIGS. 2A, 2B, and 2C illustrate various embodiments of the present system with an attached workstation.

FIG. 3 is a stylized representation of a typical B-mode image and an associated Doppler spectrum. A cross-sectional view of the ventricle and the aortic value are shown as viewed from the right atrium.

FIG. 4A is an example of a spectral Doppler graphical plot of the blood velocities during several cardiac cycles.

FIG. 4B is an example of the result of processing of the plot of FIG. 4A.

FIG. 5 is a stylized view of a B-mode ultrasound image of a valve and adjoining ventricle.

FIGS. 6A-C illustrates the basis of Doppler measurements described herein and delineates streamlined flow through a vessel, its profile through time, and the basis of the time-integral area product showing volume of flow.

FIG. 7 illustrates the delay of flow velocity relative to the associated electrocardiogram.

FIGS. 8A and 8B provides flowcharts for embodiment methods for estimating the ejection volume from spectral Doppler ultrasound signals.

FIG. 9 provides a flowchart for an embodiment method for estimating the ejection volume from a spectral Doppler ultrasound image graph.

FIG. 10 provides a flowchart for another embodiment method for estimating the ejection volume from a spectral Doppler ultrasound image graph.

### DETAILED DESCRIPTION OF THE PREFERRED EMBODIMENT

Heart failure is a disease where the heart's main function, a pump for blood, is wearing down. The heart tissue can absorb fluid, the left ventricle does not allow quick electrical conduction, becomes enlarged, does not contract well, and becomes less efficient at pumping blood. A measurement for the cardiac output (volume of ejected blood) is called the “ejection volume” or “stroke volume”. The efficiency of the heart as a pump is called the “ejection fraction” or “EF”. EF is measured as the percentage of the blood volume contained in the ventricles which is ejected with each beat of the heart. A healthy, young heart will have an EF greater than 90 (i.e., 90 percent of the ventricular blood is pumped with each heart beat); an older, sick heart in heart failure can have an EF less than 30. Heart failure leads to an extremely diminished lifestyle, and, left untreated, can be a major cause of mortality.

A new therapy to treat heart failure is bi-ventricular pacing, or “resynchronization” therapy, where both ventricles of the heart are paced with an implantable pulse generator, commonly known as an artificial pacemaker. Normal pacing for a slow heart is performed via an implanted electrode in the right ventricle. The conduction myofibers (Purkinje fibers) conduct the electrical pulse and the ventricles contract synchronously in an inward direction, resulting in blood being pumped efficiently from the heart. In heart failure, the left ventricle becomes enlarged and conduction through the tissue of the left ventricular wall often becomes slow, so that the upper part of the left ventricle conducts as much as 200 to 250 milliseconds behind the apex area of the ventricles. This leads to poor and uncoordinated contraction, and in many cases, an outward movement of the heart muscle, so that blood sloshes around rather than being squeezed out of the ventricle. Thus, an ideal site at which to place a pacing electrode in the left ventricle is in the area of slowest conduction, which can be a rather large area of the left ventricle, and may not always be the area that has the largest conduction. The problem facing physicians today is to locate the optimal site for the permanent fixation of the pacing electrode. The thrust of this invention is to provide a method and device to optimize the site of the electrode.

Within this description, the term “site” usually denotes where a specific physical feature exists anatomically within the heart, regardless of where it moves spatially over time. The term “image location” usually refers to where an anatomical site is projected into an ultrasound image frame at a specific time. An image location is measured by 2-D pixel coordinates and the moment of time within a cardiac cycle. The term “3-D location” usually refers to where in a spatial coordinate system an anatomical site is at a specific moment of time. A 3-D location is measured by 3-D coordinates and the moment of time. The term “point” may be used interchangeably with “location”. However, an image location in a specific ultrasound image frame directly corresponds to a specific anatomical site, which in turn is at a specific 3-D location at the moment corresponding to the frame. Therefore, when describing a specific ultrasound image, the terms are often blurred, and a term sometimes may infer the physical site, the site's 3-D location at the moment of image capture, and/or the site's image location at the moment. The term “position” and its related forms usually include both the concept of a 3-D location and the concept of a 3-D orientation.

A normal pacemaker electrode is ideally implanted in a site which achieves the lowest “threshold.” That is to say the site for which the lowest voltage level is needed to excite the surrounding tissue to conduct synchronously the pacing signal from the electrode. Thus, the electrode is implanted based upon merely finding the site where the lowest voltage is needed to “capture” the tissue. Placing the pacemaker electrode in the optimal site is not an easy task. Ideally, a site is chosen which optimizes the EF. Finding a site with a low threshold, while desirable, is not as important as optimizing EF. Thus, the ability to not only visualize the motion of the left ventricular wall, but also measure EF, or some form of output of the heart, such as site volume or flow rate, is highly desirable during the implantation procedure. The various embodiments use ultrasound technology to provide this ability.

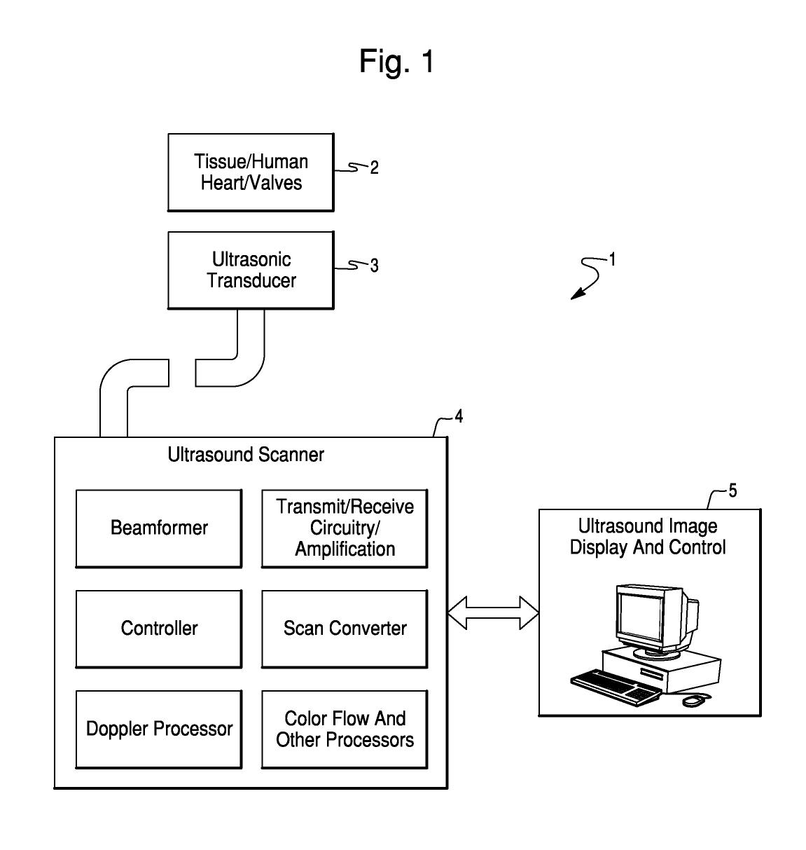

Embodiments in this specification are directed to methods and systems for measuring volumetric flow, specifically cardiac output, either with minimal intervention or input from the physician, or automatically, with the user of the system pre-specifying certain operating parameters or measurement criteria. One embodiment is in the form of hardware and/or software that exists as part of the ultrasound scanner as illustrated in FIG. 1. In such an embodiment, the system utilizes the Doppler processing capabilities of the host ultrasound scanner to obtain a time-varying signal representative of the velocity of blood flow through an area of interest. Such an area may include the inlet of the aorta from the left ventricle, or the valve in-between. The system may also utilize a view or measure of the cross-sectional area through which the flow of interest is to pass.

The Doppler signals are utilized by the processor, or any other hardware, software, or combination thereof, to calculate volume of flow through the area of interest. Doppler signals refer to shifts in the ultrasound frequency caused by the Doppler effect as blood or tissue move toward or away from the ultrasound transducer. Spectral Doppler signals can be used in combination with the dimensions of the flow field area calculate a measure of the relative amounts of flow at various velocities. Cardiac output may be computed by computing the average flow velocity at each given moment during a cardiac cycle over the area of the flow and integrating the flow over the period of one cardiac cycle.

Other embodiments also include the measuring system, either in the form of software and hardware or a combination thereof on a separate workstation/computer that is configured (i.e., programmed with software and appropriately connected to the ultrasound system) to obtain relevant data from the examining ultrasound scanner either directly or indirectly, and perform methods of gating or correlating the ultrasonic/Doppler signals (video/audio) with the electrocardiogram (ECG) of the subject being examined.

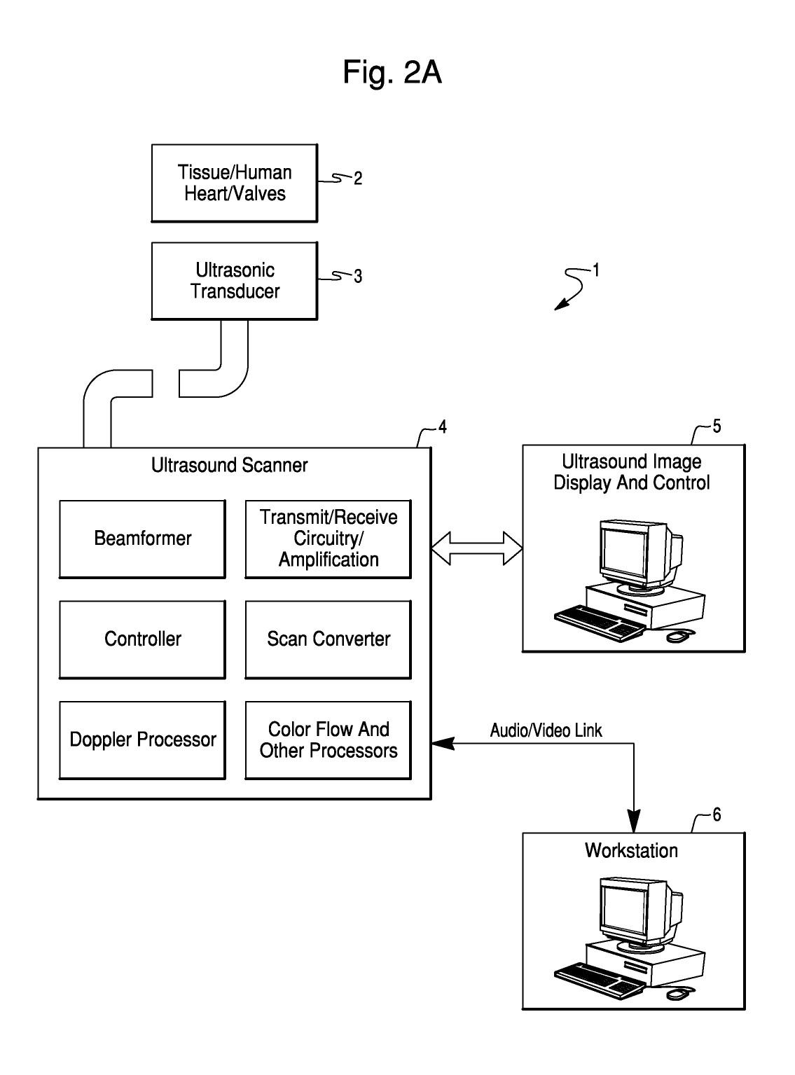

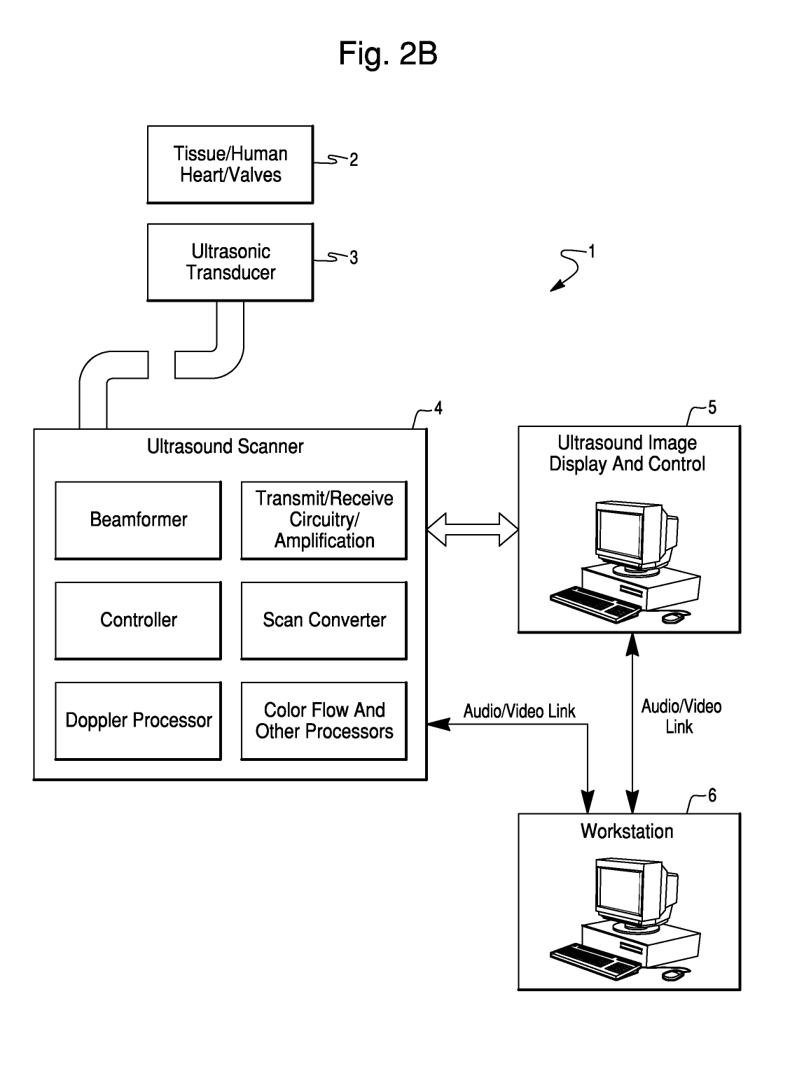

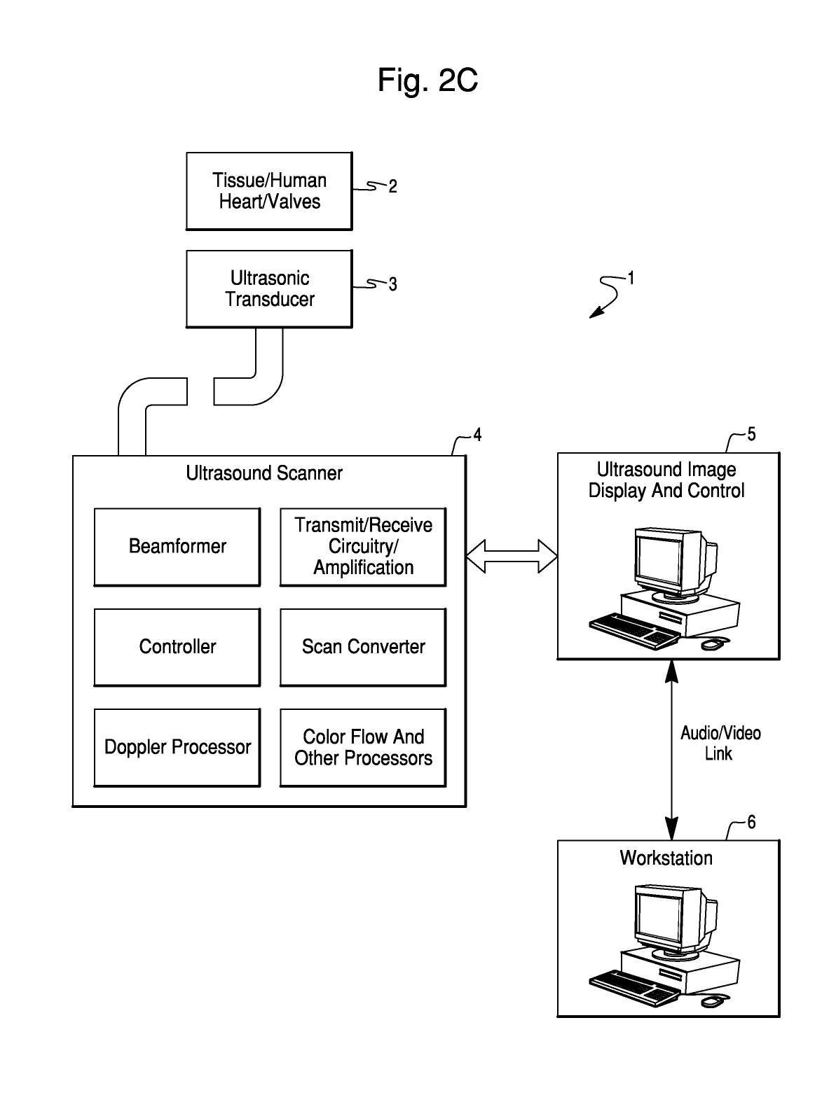

Another embodiment utilizes the Doppler audio output of the Doppler processing system/sub-system in the ultrasound machine in addition to the facilities to obtain a measure of the area of interest through which the flow is to pass, and the ECG of the subject being examined. Again, this process/system can be embodied within the hardware and/or software of the ultrasound scanner, or implemented as a workstation and/or computer separate from the ultrasound scanner with facilities to communicate either directly or indirectly with the ultrasound scanner. Such processing then uses the frequency, phase, and amplitude of the audio signals, along with the measure of the area of interest through which the flow exists, to calculate the volume of flow. Example variant system embodiments are shown in FIGS. 2A, 2B, and 2C.

A further embodiment includes methods of obtaining electrocardiogram (ECG) data from the subject being scanned to enhance the demarcation and/or separation of signals from beat to beat of the heart or to assess, either automatically or aided by a user, the condition of the cardiac system and hence the factors effecting the acquired Doppler data.

Further, an embodiment may provide a manual control or an automated detection of the delay T_{d} between the ECG R wave and the maximum measured ventricular volume, and possibly also the delay between the ECG R wave and the minimum ventricular volume. This delay measurement may be useful for pacemaker configuration and electrode placement. This delay is illustrated in FIG. 7.

Ultrasound, as an imaging tool, has been around for some time. However, imaging through the chest is very difficult in that the ribs block the view and that the depth of penetration gives poor resolution. Ideally, the ultrasound transducer should be positioned closer to the heart. An esophageal ultrasound probe has been used on many patients in an attempt to view the heart. See, e.g., Jan et. al., Cardiovasc. Intervent. Radiol., 24, 84-89 (2001). Unfortunately, the results may be less than desired since the probe must view through the esophagus and both walls of the heart, lending to less resolution in the image than desired. Intravascular ultrasound systems, although ideal in its size with thin catheters, generally utilize high frequencies which result in poor depth of penetration. X-ray or X-ray fluoroscopy may give good images of the electrode, but not of the actual tissue of the heart (most particularly the walls of the ventricle).

To overcome these problems, the embodiments preferably use an ultrasound imaging catheter designed for intracardiac use. Such an intracardiac catheter is generally sized as 10 French or less, has multiple piezoelectric elements on the transducer (e.g., 48 or 64 elements), employs lower frequencies (e.g., about 5 to about 10 MHz), and uses a phased array transducer for optimal resolution. Not only will this allow the imaging of wall motion for this specific purpose of a left ventricular electrode fixation, but will also, especially if used in conjunction with Doppler techniques, provide information to calculate measurement of cardiac output.

Such a catheter can be placed in either the right atrium of the heart or the right ventricle and easily allow viewing of the left ventricle. Another approach for viewing is from the outside of the heart, via an incision through the chest of a patient. The ultrasound display can provide a display of the measurement of cardiac output in assisting the physician with the procedure.

In addition to ultrasound imaging, a number of other items may make this implant an easier procedure, especially since many of the heart failure physicians may not have previously implanted pacemakers, may not have access to X-ray fluoroscopy, may have limited budgets for capital equipment, and may desire all discreet components used in an implantation to be accessible through one keyboard, allowing for better patient data management.

Often times the heart failure patient has a number of co-morbidities showing symptoms at the same time, such as atrial fibrillation, ventricular tachycardias, and renal failures, among others. Atrial fibrillation and ventricular tachycardia can be brought under control via electrical shock cardioversion, either internally with catheters, or externally, although with much higher energy, with patches or paddles. A cardioversion device, which could utilize the same electrodes that are otherwise introduced into the heart for pacemaker implantation, would be advantageous if also integrated with the overall electrophysiology system In this manner, inadvertent shocks could be avoided, as the trigger mechanism would come from the ventricular signal from the internal electrode. Thus, in an embodiment, the ultrasound imaging system also comprises an integral defibrillation system whereby, if needed, internal cardiac defibrillation can be implemented quickly and easily. The integrated defibrillation electrode or system may be incorporated into the ultrasound imaging catheter, attached to the ultrasound imaging catheter, or as a separate electrode system or catheter which is inserted along with the ultrasound imaging catheter.

A diagram of an embodiment is shown in FIG. 1. As shown in FIG. 1, an ultrasound imaging system 1 suitable for measuring cardiac output of a patient's heart 2, said system includes an ultrasound imaging catheter. The ultrasound imaging catheter houses at least one ultrasonic transducer 3 which utilizes piezoelectric properties to generate acoustic signals from electrical signals in order to obtain ultrasound signals. The ultrasound transducer 3 is of a type suitable for insertion into the patient's heart and is used to obtain ultrasound signals associated with an area of the patent's heart in which cardiac output is to be measured. The signals received from the ultrasound transducer 3 are fed into an ultrasonic scanner unit 4 which contains the necessary digital and/or analog electronics to generate and process ultrasound signals from the at least one ultrasonic transducer 3 to generate B-mode, M-mode, or Doppler representations of the patient's heart. These digital and/or analog electronics include, for example, a beamformer, transmit/receive circuitry and amplification circuitry, a controller unit, a scan converter, a Doppler processor and color flow as well as other processors. In addition the system includes an associated computer 5 that can generate and process the ultrasound signals in order to measure the cardiac output in the patient's heart and to measure the delay in the motion of a location on the cardiac wall with respect to the cardiac cycle.

To utilize the system of the various embodiments, a clinician may face the need to place an electrode at a desired position at or near the left ventricle of a patient's heart in order to electrically activate the left ventricle of the patient's heart using the electrode. To achieve this placement at a desired position, the user would advance the electrode to the proximity of the upper left ventricle. Before or after the electrode is in the proximity of the upper left ventricle an ultrasound imaging catheter can be placed into a position to image the left ventricle of the patient's heart. The ultrasound imaging catheter houses at least one ultrasound transducer that utilizes piezoelectric properties to generate acoustic signals from electrical signals in order to obtain ultrasound signals. Moreover, the at least one ultrasound transducer is of a type suitable for insertion into the patient's heart and capable of obtaining ultrasound signals associated with an area of the patent's heart. Once the ultrasound imaging catheter is in place it is used to image the electrode at or near the left ventricle of a patient's heart and to guide the electrode to the desired position. Once the electrode is guided into the desired position the electrode is affixed to the heart. One desired position for attachment of the electrode is the upper portion of the left ventricle (i.e., nearer the base of the heart as compared to the apex).

In an embodiment, at least one transducer has a deflecting or rotation element whereby the transducer, once positioned to image the left ventricle of the patient's heart, can be easily rotated or moved in order to image other portions of the patient's heart.

Another method for placing an electrode at a desired position on a patient's heart can be implemented in order to electrically activate the left ventricle using the electrode. In accordance with this embodiment method the ultrasound imaging catheter is placed in a position to image a ventricle of the patient's heart. The ultrasound imaging catheter placed into position houses at least one ultrasonic transducer which utilizes piezoelectric properties to generate acoustic signals from electrical signals in order to obtain ultrasound signals.

Another embodiment of the system includes an ultrasound imaging system 1 to assist in cardiac electrophysiology procedures related to a patient's heart. The system includes an ultrasound imaging catheter which houses a multi-element array transducer 3. The multi-element array utilizes piezoelectric properties to generate acoustic signals from electrical signals in order to obtain ultrasound signals. The multi-element array transducer is of a type suitable for insertion into the patient's heart and capable of obtaining ultrasound signals associated with the patent's heart. The system further includes an ultrasound scanner 4 which houses the necessary digital and/or analog electronics capable of generating and processing ultrasound signals from the multi-element array transducer to generate and display a representation of (a) the electrocardiogram of the patient's heart, (b) a real time image of the patient's heart, or (c) the cardiac output of the patient's heart. In an embodiment, the representation ultrasound signals can be displayed relative to, and compared to, a voltage conduction map of the patient's heart (i.e., a representation of the progression of electrical activation/deactivation or “action potentials” of the muscles of the heart). These electronics may include, for example, a beamformer, transmit/receive circuitry and amplification circuitry, a controller unit, a scan converter, a Doppler processor and a color flow Doppler image generator, as well as other processors.

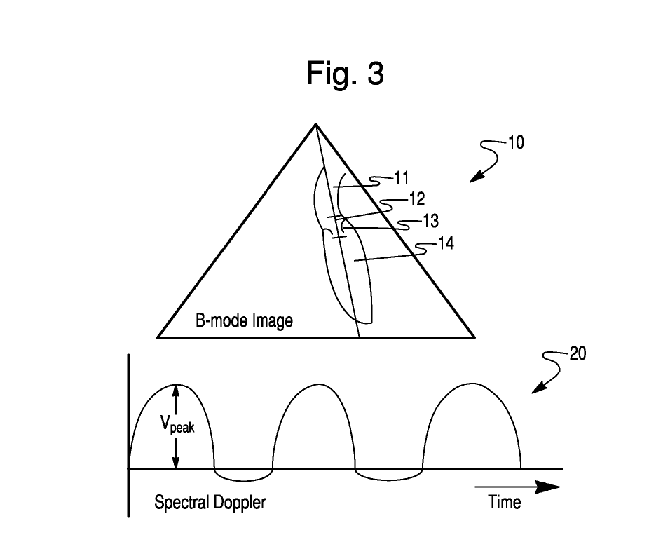

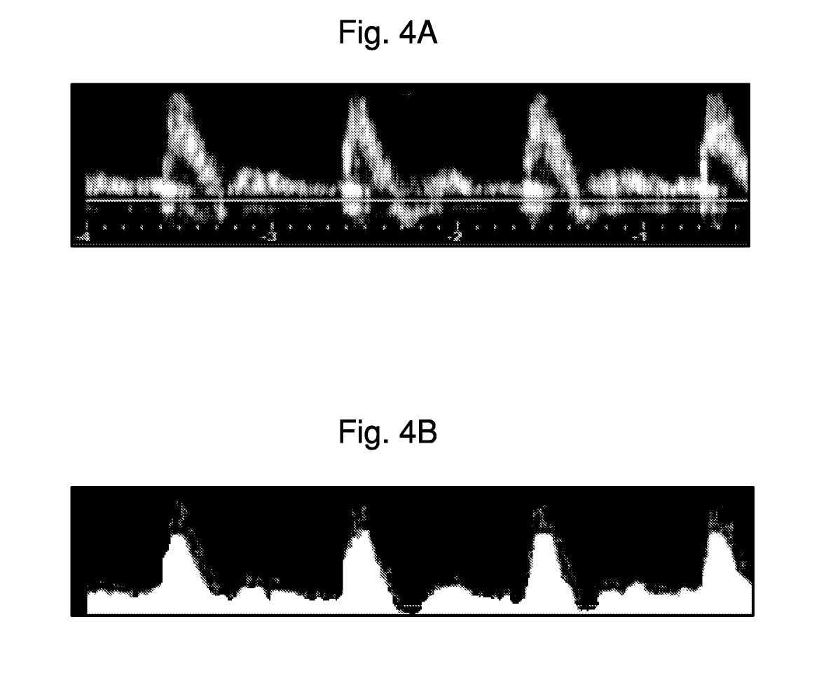

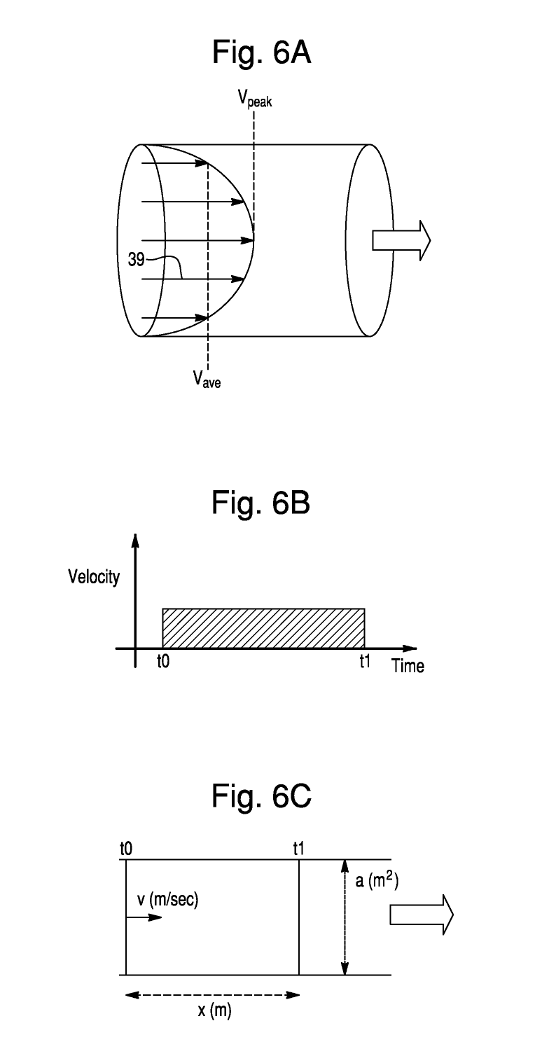

The basis of the measurement/estimation process of various embodiments is shown in FIGS. 6A-C and 7. As shown in FIGS. 6A-6C, where the Doppler process is used, the peak amplitude of the velocity profile is halved to provide the average velocity across the flow area (FIG. 6A). The velocity V is integrated (FIG. 6B) with respect to time from the start of the pulse (t0) to the end of the pulse (t1) yielding a distance x. Such integration can also include the negative signals shown in FIGS. 3 and 4A graphed below the line, which represent reverse flow during a portion of the cardiac cycle. The resulting “average stroke distance” x of this velocity-time integration is then multiplied by the cross-sectional area A of the flow to provide the cumulative ejection volume (FIG. 6C). The integration length can also be set by integrating over the complete cardiac cycle (i.e., through one complete cycle of the ECG). The spectrum in FIG. 4A can also be obtained by either frequency and/or amplitude plotting of an audio signal representing the Doppler frequency differences from the original ultrasound frequency.

An approximation to the ejection volume can be obtained by assuming that the average flow velocity at moment t is roughly one-half of the maximum of all measured velocities at moment t, where the maximum velocity Vₚₑₐₖ(t) corresponds to the largest Doppler shift (difference) frequency at time t:
Vₑⱼₜ=A·∫Vₚₑₐₖ(t)/2 dt Eq. 1
where:

Vₑⱼₜ=approximate absolute ejection (stroke) volume;

A=cross sectional area of flow; and

Vₚₑₐₖ(t)=the peak velocity at time t.

Note that the value of cross-sectional area A for a given individual may be relatively constant, at least for certain specific imaging sites, such as the inlet to the aorta. Therefore, for the purpose of relatively comparing the effectiveness of implanting an electrode at each of two or more cardiac locations, the exact value of A may be ignored because it is common to all the measurements. The value of A may be left symbolic, left undefined, set to a typical value, or simply set to 1 for all computations for electrode locations being compared relatively.

An alternative to using one-half the peak velocity to approximate the average velocity is to perform a more comprehensive computation of the average velocity. At any given moment in the cardiac cycle, the spectral Doppler signal consists of a spectrum of frequency shifts, each corresponding to a velocity of blood within a specific small volume through a portion of the cross-sectional area through which the blood is flowing. The more blood flowing at each given velocity, the greater the amplitude of the reflected ultrasound echo signal at the frequency corresponding to the given velocity. Therefore, a useful approximation to the average velocity is the amplitude-weighted average of all the velocity components represented in the spectrum at time t. That is, the average velocity can be calculated by integrating the product of each velocity v(f) corresponding to each Doppler frequency shift f in the spectrum and the amplitude E(f) of the echo at that frequency and dividing by the total amplitude:
v_{avg}(t)=∫_{f>0}v(f,t)E(f,t)df/∫_{f>0}E(f,t)df Eq. 2a
In practice, the spectrum will be represented by discrete, measured frequency shifts measured at small intervals and at discrete times tᵢ. Therefore, the integration may be approximated by this summation:
v_{avg}(t)=Σ_{i=1, . . .} v(fᵢ)·E(fᵢ,tᵢ)/Σ_{i=1, . . .} E(fᵢ,tᵢ) Eq. 2b
where:

v_{avg}(t)=average velocity at time t;

v(fᵢ,tᵢ)=velocity corresponding to frequency shift fᵢ at time tᵢ; and

E(fᵢ,tᵢ)=amplitude of echo at frequency shift fᵢ at time tᵢ.

Because of the direct relationship of frequency shift to velocity, we can alternatively first compute the average frequency shift F_{avg}(t) and then convert F_{avg}(t) to the corresponding average velocity v_{avg}(t):
F_{avg}(t)=Σ_{i=1, . . .} fᵢ·E(fᵢ,t)/Σ_{i=1, . . .} E(fᵢ,t) Eq. 2c

Because the Doppler frequency shift F_{avg}(t) corresponds to a velocity, F_{avg}(t) may be converted into an average velocity by:
v_{avg}(t)=C·F_{avg}(t) Eq. 2d
where C=a proportionality constant reflecting the speed of sound in blood and the frequency of the emitted ultrasound. More particularly, assuming the transducer is still so the only complement of relative velocity is from the blood reflecting ultrasound, then the velocity of the blood along the line between the transducer and the blood (i.e., toward or away from the transducer) will be:
V_{b}=½ S_{b}(Δf/fₑ)
where:

V_{b}=velocity of the blood reflecting sound;

S_{b}=speed of sound in blood;

Δf=frequency of received sound minus the frequency of emitted sound; and

fₑ=frequency of the emitted sound.

The factor of one-half is due the fact that the ultrasound is emitted by the transducer so the received ultrasound echo includes two velocity components. Since the speed of sound in tissue is approximately a constant (˜1480 m/s), and the emitted ultrasound frequency is constant (e.g., in the range of 5-10 MHz), the speed of blood toward or away from the transducer may be expressed as a constant times the frequency shift (i.e., difference between received and emitted frequencies), one-half the speed of sound in blood times the percentage frequency shift (Δf/fₑ), or as a constant times received frequency minus another constant. Corrections for off-axis measurements of blood flow can then be made by applying an appropriate trigonometric factor.

The value Δx(t)=v_{avg}(t)·Δt(t) represents the mean distance the blood has flowed during time interval Δt(t), where Δt(t) is the time interval corresponding to v_{avg}(t). The sum over all intervals dt in a cardiac cycle may be called the average per-stroke ejection flow distance x:
x=Σₜ v_{avg}(t)·Δt(t) Eq. 3
In effect, it is an approximation of the average distance traveled by a blood cell. In the common special case where Δt(t) is constant for the ultrasound system, for an ultrasound imaging session or for a set of ultrasound images, then Δt(t) may be ignored, and x is simply the product of the length of time T=Σₜ Δt(t) of the cardiac cycle times the average velocity over all the intervals t of the cardiac cycle:
x=T·v_{avg}. Eq. 4a
where v_{avg}=average of all v_{avg}(t) for all t in T.

If the cross-sectional area A of the flow is known, then the ejection (stroke) volume may further be computed using x as:
V=A·x Eq. 4b

It is worth noting that in the equations and discussion above the emitted frequency is a known constant, and therefore the methods may use the received sound frequency, the frequency shift (i.e., the difference between received and transmitted frequencies) or fractional frequency shift (e.g., percentage shift) in the received sound with simple arithmetic transformations. Thus, references to frequency and frequency shift may be used interchangeably as would be appreciated by one of skill in the art. Accordingly, reference is made to the general term “Doppler frequency shift data” herein to encompass the various measures of Doppler shift.

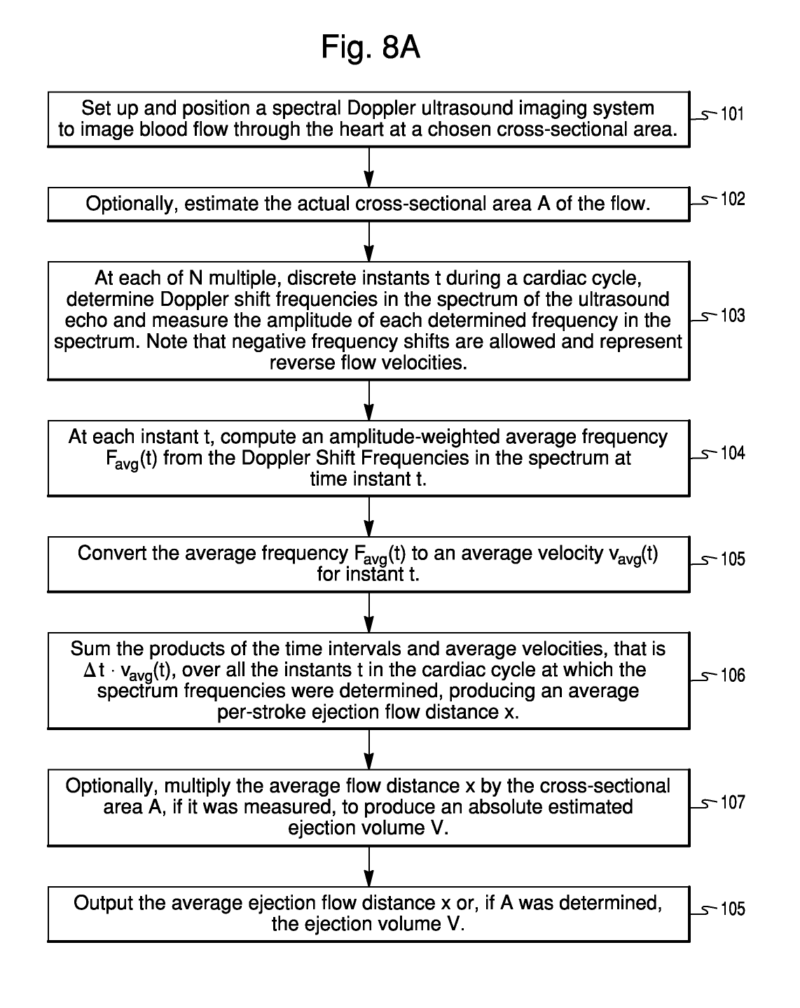

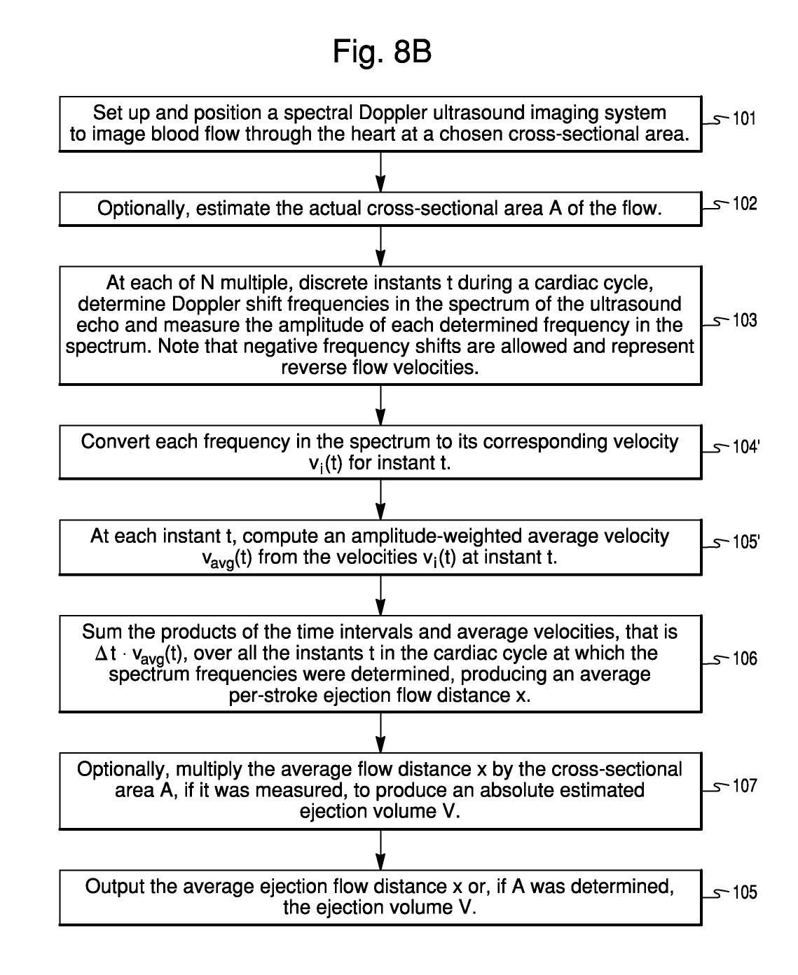

An example embodiment of the preceding equations using Doppler flow velocity measurements is provided as a flowchart in FIG. 8A. As described in FIG. 8A, the clinician sets up and initializes a spectral Doppler ultrasound imaging system, including positioning the ultrasound probe to view a cross-section of blood flow through a chamber of the heart or through an adjoining vessel such as the aorta: step 101. To compute an estimate of the absolute volume, as opposed to a relative value to be used for comparisons, the cross-sectional area of the flow is be measured: step 102. This measurement may be performed manually by the clinician, automatically by the imaging system, or semi-automatically, such as directing the clinician to mark a number of image locations on edges of the image of the cardiac or vessel wall.

The spectral Doppler system then measures the spectrum of the Doppler echo frequency shifts at each of a number of moments t during one or more cardiac cycles: step 103. These frequency shifts correspond to various velocities within the imaged portion of the blood flow. There is a direct relationship between the frequency shift and the velocity—governed by the propagation speed of sound in blood.

The amplitude-weighted average frequency F_{avg}(t) of the frequencies appearing in the spectrum at time t is computed as in Eq. 2c: step 104. The average frequency shift F_{avg}(t) at time t in the cardiac cycle can be converted into an average velocity v_{avg}(t) by multiplying by a constant based on the speed of sound in blood: step 105. By summing the product of the time intervals and these velocities v_{avg}(t) over all the times t in a cardiac cycle for which the spectra were measured, the average per-stroke ejection flow distance x is computed as in Eq. 4a: step 106. If the cross-sectional area A is known (e.g., measured in step 102), then an estimate of the ejection volume V is A time x: step 108. See Eq. 4b. Otherwise, x may be used as a relative measure cardiac output for comparison purposes, for example of two or more sites for locating a pacemaker electrode.

The steps of FIG. 8A need not necessarily be performed in the order listed. For example, step 102 may be preformed at any time before step 107.

A variant embodiment illustrated in FIG. 8B may include first converting the frequencies (step 104′) in the spectrum measured at time t into velocities (i.e., by determining the frequency shift and multiplying that value times the constant based on the speed of sound in blood) and then computing the amplitude-weighted average (step 105′) of the velocities, as in Eq. 2b. In effect this variant embodiment reverses the order of steps 104 and 105 of FIG. 8A.

An alternative embodiment may perform a simplified graphical version of the above velocity-time summation, as follows: The ultrasound system may display the spectral response over one or more cardiac cycles as a pixel-based graphical image, or plot. That is, each pixel column of the spectral Doppler image represents a specific moment in the cardiac cycle, and the row height of a pixel in the column represents a specific frequency or rather a specific narrow band about the frequency. The intensity of the pixel represents the amplitude of the signal having the specific frequency at the instant in time t. FIG. 4A is an example of such a spectral Doppler image for several cardiac cycles. The average velocity v_{avg}(t) at any instant t may therefore be approximated by finding the intensity-weighted average row height of all the pixels in the column corresponding to that instant t in the graphical plot of the spectral Doppler frequency spectrum. Let F(t) be the weighted average frequency at time t. F(t) may be computed for each of the columns in a cardiac cycle image, which columns are all the discrete instants t for which there is a measured ultrasound spectrum. That is:
F_{avg}(t)=Σᵢ Wᵢ(t)·Fᵢ(t)/Σᵢ Wᵢ(t) Eq. 5
where:

F_{avg}(t)=weighted average frequency shift at time instant t (column);

i=pixel height (row) in the spectral Doppler display;

Wᵢ(t)=pixel intensity of at pixel row and time (column) t; and

Fᵢ(t)=spectral Doppler frequency shift corresponding to row i.

Note that the pixel intensity Wᵢ(t) corresponds proportionately to the echo intensity of Fᵢ(t). Because the Doppler frequency shift F(t) corresponds to a velocity, F_{avg}(t) may be converted into an average velocity v_{avg}(t)=C·F_{avg}(t), where C is a proportionality constant reflecting the speed of sound in blood.

Let R(t) be the integer pixel row which most corresponds with F_{avg}(t), where R(t)=0 is the row corresponding to a zero Doppler frequency shift. More directly,
R(t)=round(Σᵢ Wᵢ(t)·Rᵢ(t)/Σᵢ Wᵢ(t)) Eq. 6
where:

R(t)=nearest integer row coordinate representing the average frequency;

Rᵢ(t)=the row number i of pixel i in column t; and

Wᵢ(t)=pixel intensity of at pixel row and time (column) t.

Note also that reverse flow velocities may be represented by negative values of R(t) or Rᵢ(t).

In an embodiment, a discrete approximation of the velocity-time integral Eq. 1 is approximately proportional to the total number of pixels in the image which are below or equal to row R(t) and above or equal to zero for all t running through one cardiac cycle. If F_{avg}(t) is negative, that is R(t) is negative in a column corresponding to t (indicating a reverse average flow in certain pathological cases), then the number of pixels between R(t) and zero may be subtracted. The time t, or rather the corresponding column, runs through one cardiac cycle, which may be defined by the peak of the R wave of an ECG signal (if available) or may be defined by the columns between two adjacent suitably chosen local peak values of R(t), for example. The peaks may be chosen to be some minimum time apart (such as 200 milliseconds), be the maximal value within a surrounding time interval (such as 200 milliseconds), and be separated by values of R(t) which drop below some fraction of the peak value. Where one or more columns contains only a weak spectral data, such as total column pixel intensity below some threshold, an embodiment may linearly interpolate using the R(t) values of the nearest columns on either side with reliable data.

An embodiment may visually display the pixel counting technique by graphically overlaying the spectral Doppler image with the pixels R(t), possibly in a color contrasting to the image. Alternatively, to show the total flow more explicitly, all the pixels at or below R(t) and above zero may be overlaid over the image or displayed separately. FIG. 4B shows the result performed for the example spectral Doppler image of FIG. 4A. Note that at a few times (in a few columns) the spectral information is nearly missing, presumably because of ultrasound artifacts, not representing literal conditions in the heart. In such cases R(t) may be interpolated from surrounding columns in order to avoid sudden discontinuities due to artifacts and noise. Alternatively, the results from a number of cardiac cycles may be combined in an average spectral Doppler image since averaging over many cycles should fill in random artifacts and noise.

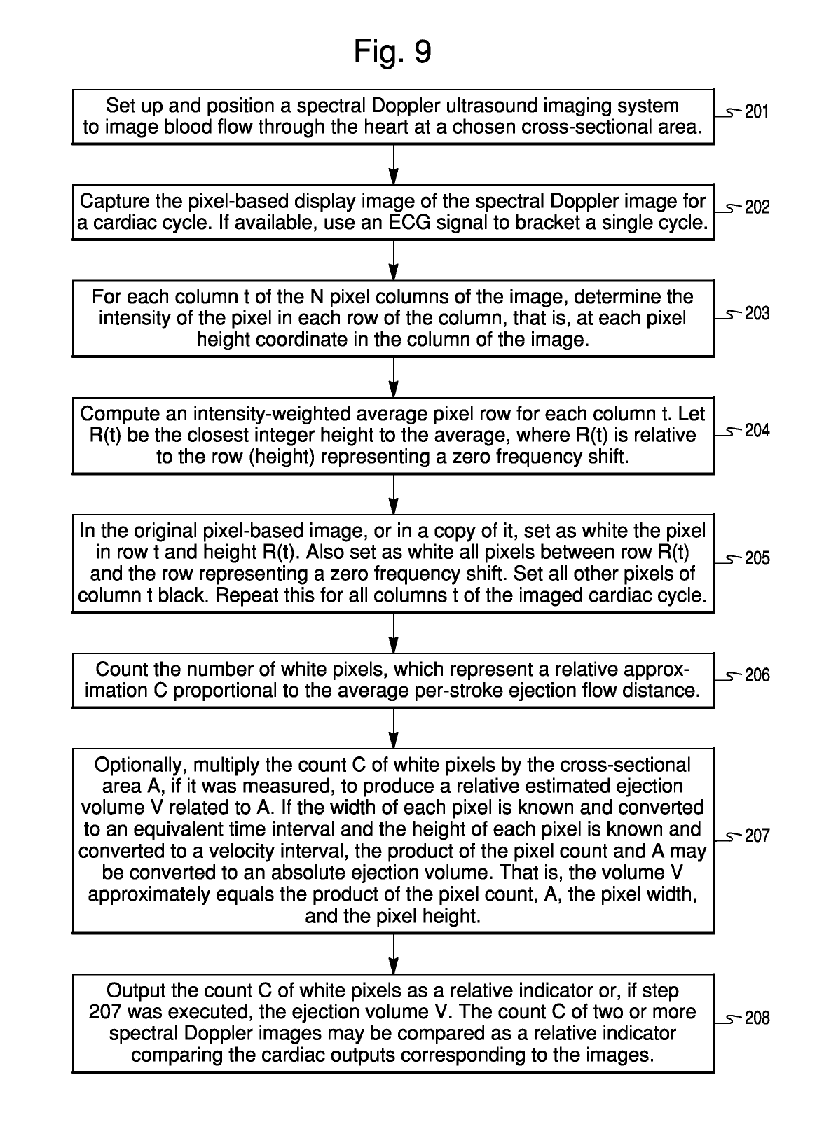

FIG. 9 presents an embodiment of a method which uses the pixel-based display image itself as a basis for approximating a relative measure of cardiac output. The clinician sets up and positions a spectral Doppler ultrasound imaging system, including positioning the ultrasound probe to view a cross-section of blood flow through a chamber or valve of the heart or through an adjoining vessel such as the aorta: step 201. To compute an estimate of the absolute volume, as opposed to a relative value to be used for comparisons, the cross-sectional area of the flow is measured: step 202. This measurement may be performed manually by the clinician, automatically by the imaging system, or semi-automatically, such as directing the clinician to mark a number of image locations on edges of the image of the cardiac or vessel wall. For relative comparisons of the heart output measurements where the area A is known to be invariant (i.e., the same for all measurements), the measurement of area A can be skipped.

The spectral Doppler system then measures and displays a graphical plot of the spectrum of the Doppler echo frequency shift at each of a number of moments t during one or more cardiac cycles: step 203. Typically, each moment t of a cardiac cycle corresponds with a column of pixels in the plot, and the row, or height coordinate, of each pixel in a column corresponds to a narrow band of Doppler shift frequencies in the spectrum. The frequency shifts correspond to various velocities in small volumes within the imaged portion of the blood flow. There is a direct, known relationship among the pixel height within a column, the frequency shift, and the velocity. The intensity of a plot pixel is proportional to the amplitude of the echo frequencies within the narrow band of frequencies mapped to the row height (coordinate) of that pixel.

The pixel-intensity-weighted average pixel height R(t) within a column (moment) t is computed as in Eq. 6: step 204. If desired, R(t) may be converted into an average velocity v_{avg}(t) by determining the center frequency shift of the band R(t) and multiplying by a constant based on the speed of sound in blood. The system may then select the pixel which is nearest to (i.e., corresponds most closely to) the center frequency shift of that band. The system then may mark the selected pixel in each column t at row R(t), such as by setting the pixel to white: step 105. Next, all pixels in column t below the selected pixel are also marked (e.g., by setting the pixels to white), and all other pixels in column t are disregarded or set as unmarked, such as by setting those pixes to black. This pixel selecting and marking may be done on the original displayed graphical image plot itself or on a separate graph. The total count of the marked (white) pixels for all the times t in a cardiac cycle provides a measure of the relative output of the heart in one cycle: step 106. Knowing the time interval Δt of each column and knowing the velocity interval for each pixel row allows conversion of the pixel count to an approximation of the average per-stroke ejection distance x. If the cross-sectional area A is known (e.g., measured in step 202), then an estimate of the ejection volume V is A time x: step 208. Otherwise, x or simply the marked pixel count may be used as a relative measure of cardiac output for comparison purposes, for example of two or more sites for a pacemaker electrode.

The steps of FIG. 9 may be performed in a different order within alternative embodiments or be constituted otherwise to produce similar results. For example, the determination of area A, if performed, may be executed at any time prior to step 208. Further, the pixel at row R(t) for each column t may be marked by setting its color to red, while the pixels in column t below the pixel may be marked blue. The pixel count would include both red and blue pixels. Alternatively, the pixels may be marked in ways that are not visible at all.

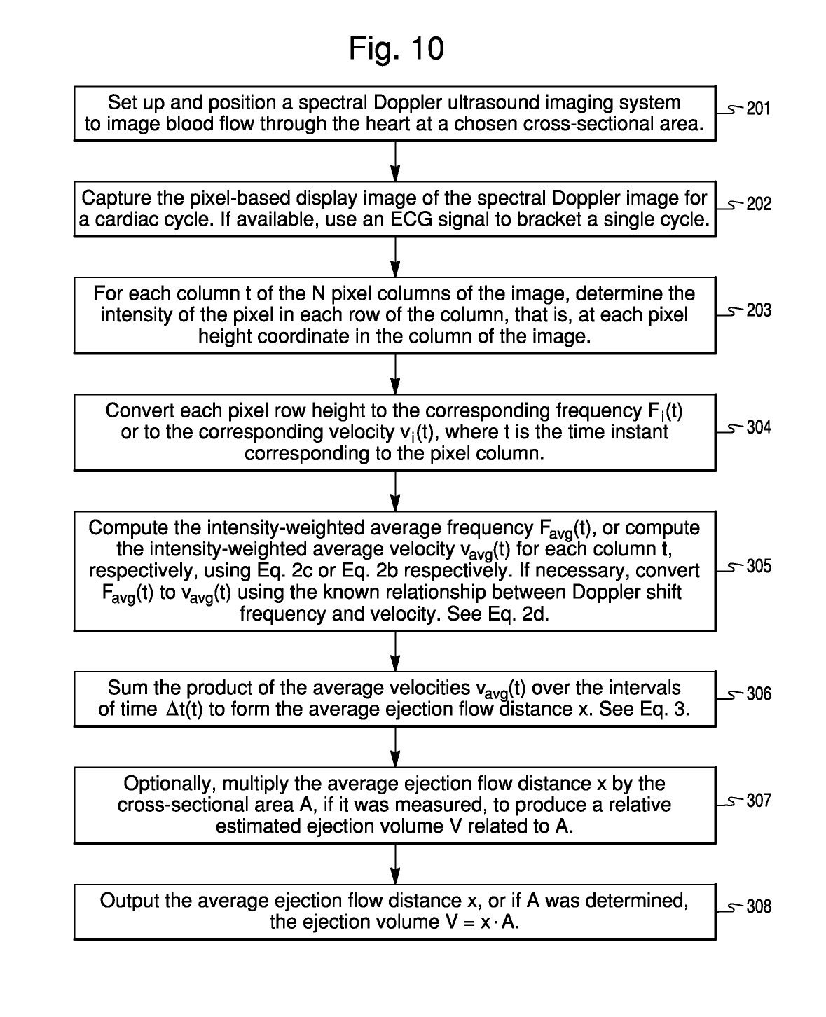

FIG. 10 shows a variant embodiment which also begins with the pixel-based graph of the Doppler spectrum over the course of at least one cardiac cycle. Steps 201, 202, and 203 of FIG. 10 are like steps 201, 202, and 203 of FIG. 9. In FIG. 10, the method converts each pixel row (height) i into a frequency Fᵢ(t), directly into a velocity vᵢ(t), or derives vᵢ(t) from Fᵢ(t) in each column, which represents time instant t: step 304. The average velocity v_{avg}(t) at time instant t is computed from vᵢ(t), if known: step 305. Alternatively, F_{avg}(t) is computed from Fᵢ(t) and then converted to v_{avg}(t) using the known relationship between Doppler frequency shift and velocity. Using Eq. 3, the products of the time interval at t and the corresponding v_{avg}(t) are summed over a cardiac cycle: step 306. Steps 307 and 308 are similar to the last steps of FIGS. 8A, 8B, and 9.

When the cross-sectional areas of flow corresponding to two spectral Doppler images differ, the velocity-time integral cannot be used without including the appropriate area factor A. However, the pixel-count technique may still be applied. Each area A may be estimated using, for example, the built-in video image calipers on a B-mode or M-mode image corresponding with each spectral Doppler image. The value of each pixel-count approximation of the velocity-time integral is multiplied by the corresponding area to obtain an estimate of the absolute ejection volume.

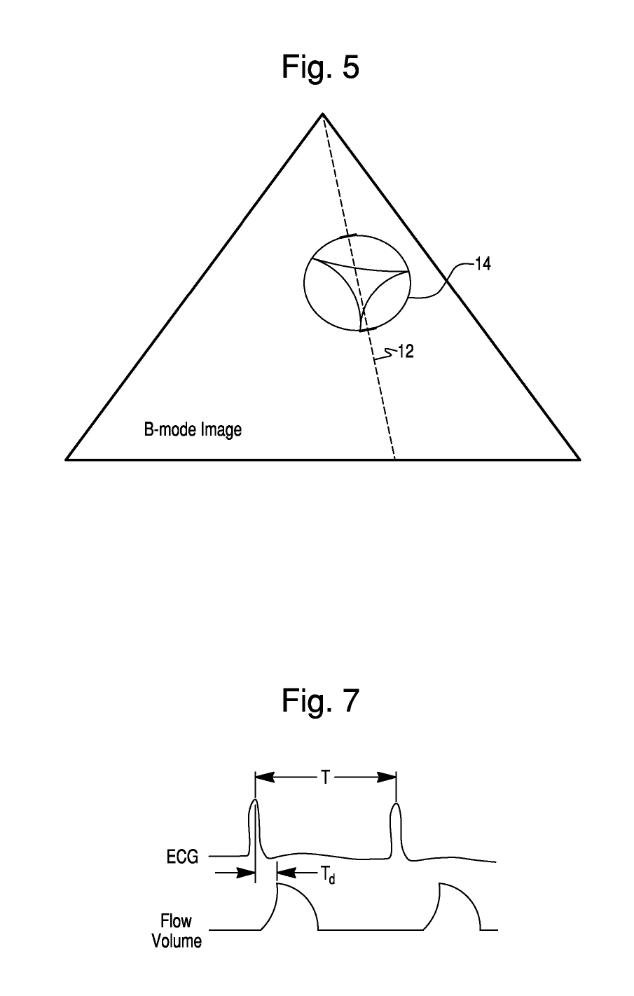

An embodiment may be in the form of hardware and/or software that exists as part of the ultrasound scanner (FIG. 1). In such an embodiment, the system can utilize the Doppler processing capabilities of the host ultrasound scanner 4 to obtain a time-varying signal representative of the velocity of flow through an area of interest. Such an area could include the inlet of the aorta from the left ventricle, or the ventricular valve 13 (FIG. 3). The system also may utilize a view and measure of the cross-sectional area 14 through which the flow of interest is to pass (FIG. 5).

The Doppler system outputs the spectral Doppler frequency shift information 20, which is indicative of the velocity of flow through the volume of interest (as shown in FIG. 4A) by means of showing a spectrum (which in some embodiments can be obtained in analog or digital format from the machine). or outputs spectrum data for frequency shift data (i.e., difference between the measured and emitted frequencies). Such a spectrum can be obtained either by obtaining a longitudinal sectional view of the flow axis at any angle (as represented in FIG. 3), or by obtaining a cross sectional view of the flow conduit (FIG. 5). Such calculations of flow/area can compensate for the angle of measurement using a cosine of the angle with respect to actual plane correction. For conditions where the flow is perpendicular to the sample volume of the Doppler system, other estimation techniques such as “Transverse Doppler,” which utilizes the Doppler bandwidth to assess flow at flow to beam angles close to 90 degrees, can be utilized. See Tortoli et al., Ultrasound Med. Biol., 21, 527-532 (1995). This Doppler signal can also exist as an audio signal (again, either in analog or digital format) as a frequency and/or amplitude modulated signal that is indicative of the spectrum and hence the flow velocity through the area of interest. If the signal is an audio signal and represents the Doppler frequency shift spectrum, spectral analysis may be applied by a processor as in a spectral Doppler system. The signal may further include ECG signals (again, in analog or digital format).

Alternatively, a semi-automated process may be employed wherein the system (either on the ultrasound machine or a separate computer connected to or otherwise configured to receive Doppler spectrum data from the ultrasound machine) automatically integrates the velocity curve, with or without the help of an ECG input, while the user inputs the cross-sectional area of interest of the orifice through which the flow passes.

Alternatively, a fully automated process may be employed wherein the system (or a separate computer connected to or otherwise configured to receive Doppler spectrum data from the system) prompts the user to obtain particular views of the anatomy of interest and demarcate specific points and the system then processes the data as above with the system automatically determining the area (or region) of interest.

The system can automatically integrate the velocity curve from beat to beat, and output the stroke volume in any sort of display, having obtained the cross sectional area using the techniques mentioned herein. Of course, various combinations and/or modifications of these techniques can be used if desired and depending on the particular application and/or patient.

An embodiment which estimates the absolute or relative stroke volume or ejection fraction over a single cardiac cycle can be further enhanced by averaging the per-cycle stroke volume or the per-cycle ejection fraction over multiple cycles. Besides providing a more representative average value, measurements over a number of cycles allows the computation of the statistical variation or standard deviation. The standard deviation then provides an indication of the variability of the multiple cycles' values from the mean value which may be used for diagnostic purposes, such as a measure of the arrhythmia.

A fully automated process may be employed wherein the system prompts the user to obtain particular views of the anatomy of interest and to demarcate specific cardiac sites for tracking, such as on the wall of the ventricle. The system then processes the data as above with the system internally tracking the sites and computing the cross-sectional area. The system can automatically measure the stroke volume, with data computed from any of the above described methods, and output the stroke volume in any sort of display, having obtained the cross sectional area using the cardiac sites.

Another embodiment is in the form of a computer (e.g., a workstation) operating software that exists separate from the ultrasound scanner console or workstation with means to communicate data, video and/or audio and/or other signals between the ultrasound scanner and/or the display computer/system (as exemplified in FIGS. 2A, 2B, and 2C). Communication between such a separate computer and the ultrasound scanner could include ultrasound data, spectral Doppler frequency spectrum data, video, audio, and/or any ECG signals in digital and/or analog format. The processing describe herein can then be performed either partially or entirely on the separate computer.

Another embodiment can include hardware and/or software separate from the ultrasound scanner, in the form of a workstation 6 wherein there exists a mode of communication, either analog or digital, between the workstation 6 and the ultrasound scanner 4 or catheter 3. See FIGS. 2A and 2B. Cabling from the ultrasound machine 4 to the catheter 3 (especially with a multi element array catheter) and from the catheter proximal connector to the catheter transducer housed at the distal tip can be expensive. To reduce cost, the ultrasound machine 4 can be moved adjacent to the patient 2, thereby allowing a relatively short cable to be used to attach the catheter. In some cases, however, this may be impractical since most catheter rooms are sterile or semi-sterile environments and, thus, the ultrasound machine may be some distance from the patient's bedside. Thus, a connecting cable which is reusable (and probably non-sterile) is desirable, as opposed to the catheter itself, which is sterile and usually not re-usable. It would be desirable if this connecting cable could be used as a universal cable in that it could be used with many ultrasound machines. While many ultrasound machines have a standard 200 pin ZIP connector, most ultrasound machines do not have patient isolation means built in to the degree necessary for percutaneous catheter use. Therefore, in another embodiment as shown in FIG. 8, the system of this invention employs a connector cable with an isolation box 7 that is external to the ultrasound machine 4 itself. Preferably the isolation box, which houses a plurality of isolation transformers, is relatively small so that it can be placed easily on or near the patient's bed. Such a cable can accommodate all operational communication between the catheter 3 and the ultrasound machine and/or the appropriate computer workstation.

In still another embodiment, the ultrasonic catheter further comprises a temperature sensing and/or control system. Especially when used at higher power (e.g., when using color Doppler imaging) and/or for lengthy periods of time, it is possible that the transducer, and hence, the catheter tip, generate heat that may damage tissue. While computer software can be used to regulate the amount of power put into the catheter to keep the temperature within acceptable ranges, it is also desirable to provide a temperature sensing means as well as a safety warning and/or cut-off mechanism for an additional margin of safety. Actual temperature monitoring of the catheter tip is most desirable, with feedback to the computer, with an automatic warning or shut down based upon some predetermined upper temperature limit. The system can be programmed to provide a warning as the temperature increases (e.g., reaches 40° C. or higher) and then shut off power at some upper limit (e.g., 43° C. as set out in U.S. FDA safety guidelines). To monitor the temperature at or near the tip of the catheter (i.e., in the region of the ultrasound transducer), a thermistor may be used. The temperature at the tip of the catheter can be continuously monitored via appropriate software. Although the software can also provide the means to control the power to the catheter in the event that excessive temperatures are generated, it may also be desirable to have a back up, shut off, or trip mechanism (e.g., a mechanical or electrical shut off or tripping circuit).

In a further embodiment, the ultrasound system, isolation box and temperature monitoring/cutoff circuits may be packaged as a combined unit which can be placed close to the patient and eliminate or shorten some of the cables required for a system comprised of separate components.

Of course, various combinations and/or modifications of these techniques and systems can be used if desired and depending on the particular application and/or patient.

It is to be understood, however, that even though numerous characteristics and advantages of various embodiments have been set forth in the foregoing description, along with details of the structure and function of the invention, the disclosure is only for illustrative purposes. Changes may be made in detail, especially in matters of shape, size, arrangement, storage/communication formats and the order of method steps within the principles of the invention to the full extent indicated by the broad general meaning of the terms in which the appended claims are expressed.

What is claimed is:

1. An ultrasound imaging system suitable for estimating cardiac output of a patient's heart, comprising:
an intracardiac ultrasound system configured to
receive ultrasound signals from an intracardiac ultrasound transducer when positioned within the patient's heart, and
output Doppler frequency shift data at each of a plurality of sub-second intervals of received ultrasound signals for at least one cardiac cycle; and
a computer configured to:
receive the Doppler frequency shift data;
process the Doppler frequency shift data at each of the plurality of sub-second intervals to compute an amplitude-weighted average frequency shift at each of the plurality of sub-second intervals;
convert the amplitude-weighted average frequency shift to an average velocity for each of the plurality of sub-second intervals;
sum the average velocities of the plurality of sub-second intervals over a cardiac cycle; and
estimate the cardiac output from the sum of the average velocities and the durations of the plurality of sub-second intervals.

2. The ultrasound imaging system of claim 1, wherein the computer is further configured to:
obtain a cross-sectional area of a region through which the Doppler frequency shift data are measured; and
compute an ejection volume based on the cross-sectional area, the sum of the average velocities, and the durations of the plurality of sub-second intervals.

3. The ultrasound imaging system of claim 1, wherein the computer is a component of the intracardiac ultrasound system.

4. An ultrasound imaging system suitable for estimating cardiac output of a patient's heart, comprising:
an intracardiac ultrasound imaging catheter including at least one ultrasound transducer;
an ultrasound scanner configured to receive ultrasound signals from the intracardiac ultrasound imaging catheter, the ultrasound scanner comprising a spectral Doppler processor configured to generate ultrasound amplitude and Doppler frequency shift data at each of a plurality of sub-second intervals spanning at least one cardiac cycle based on the received ultrasound signals; and
a computer configured to:
process the Doppler frequency shift data at each of the plurality of sub-second intervals;
compute a set of velocities based on the Doppler frequency shift data at each of the plurality of sub-second intervals of the at least one cardiac cycle;
compute an amplitude-weighted average velocity over the cardiac cycle; and
estimate the cardiac output from the amplitude-weighted average velocity.

5. The ultrasound imaging system of claim 4, wherein the computer is further configured to compute an ejection volume based on the amplitude-weighted average velocity and a cross-sectional area measure of a region corresponding to the Doppler frequency shift data.

6. The ultrasound imaging system of claim 4, wherein the computer is a component of the ultrasound scanner.

7. An ultrasound imaging system suitable for estimating cardiac output of a patient's heart, comprising:
an intracardiac ultrasound imaging catheter including at least one ultrasound transducer;
an ultrasound scanner configured to receive ultrasound signals from the intracardiac ultrasound imaging catheter at each of a plurality of sub-second intervals spanning at least one cardiac cycle, the ultrasound scanner comprising a spectral Doppler processor configured to
generate Doppler frequency shift data at each of sub-second intervals spanning the at least one cardiac cycle, and
generate a pixel-based plot of the Doppler frequency shift data, wherein the pixel-based plot comprises a plurality of columns of pixels; and
a computer configured to:
receive the pixel-based plot of the Doppler frequency shift data;
select a representative pixel in each column of the plurality of columns of pixels;
in each one of the plurality of columns, mark all pixels in the one column equal to or below the representative pixel selected for the one column and disregard all pixels in the one column above the representative pixel selected for the one column; and
sum the marked pixels as an estimated measure of cardiac output.

8. The ultrasound imaging system of claim 7, wherein the representative pixel is selected as the pixel closest to an amplitude-weighted average pixel value for all pixels within the one column of the plurality of columns.

9. The ultrasound imaging system of claim 7, wherein the computer is further configured to compute the measure of cardiac output based on the count of the marked pixels and a cross-sectional area measure of a region corresponding to the Doppler frequency shift data.

10. The ultrasound imaging system of claim 7, wherein the computer is a component of the ultrasound scanner.

11. A method for measuring cardiac output of a patient's heart, comprising:
positioning an intracardiac ultrasound imaging catheter in or near the patient's heart;
obtaining a pixel-based plot of Doppler frequency shift data using ultrasound data obtained from the intracardiac ultrasound imaging catheter at each of a plurality of sub-second intervals spanning at least one cardiac cycle, wherein the pixel-based plot comprises a plurality of columns of pixels;
processing each one column of the plurality of columns to select a representative pixel for the one column, wherein the representative pixel is selected as the pixel in the one column which is closest to a pixel-intensity-weighted average for the one column;
marking each representative pixel and all pixels between the representative pixel and a pixel row representing a zero Doppler shift frequency in each of the plurality of columns; and
counting the number of marked pixels as a measure of cardiac output.

12. The method of claim 11, further comprising:
obtaining a cross-sectional area of a region through which the Doppler frequency shift data are measured; and
computing a measure of the cardiac output based on the cross-sectional area and the count of marked pixels.
